(19) United States
(12) Patent Application Publication
(10) Pub. No.: US 2026/0334998 A1
(43) Pub. Date: Oct. 29, 2026

(54) HEART VALVE LEAFLET GRABBING AND SLITTING DEVICE

(71) Applicant: KONINKLIJKE PHILIPS N.V., EINDHOVEN (NL)

(72) Inventor: Thomas John MCPEAK, MAPLE GROVE, MN (US)

(21) Appl. No.: 19/486,323

(22) PCT Filed: Jun. 24, 2024
(86) PCT No.: PCT/EP2024/067587

§ 371 Date: Nov. 20, 2025

Related U.S. Application Data
(60) Provisional application No. 63/523,715, filed on Jun. 28, 2023.

(51) Int. Cl. A61B 18/08; A61B 18/00; A61B 18/10

(52) U.S. Cl. CPC A61B 18/085; A61B 18/10; A61B 2018/00077; A61B 2018/00184; A61B 2018/00351; A61B 2018/00601

(57) ABSTRACT

A tissue gripping and slitting device includes a catheter configured to be positioned within a body lumen of a patient, and a gripping assembly coupled to the catheter. The gripping assembly includes a first jaw, a second jaw, and a hinge mechanism coupled to the first jaw and the second jaw and configured to rotate the first jaw relative to the second jaw between an open state and a closed state. The device also includes a cutting assembly coupled to the gripping assembly and including at least one conductor and a cutting element configured to be energized by the at least one conductor.

### TECHNICAL FIELD

The subject matter described herein relates to cardiac valve replacement and, in particular, to the reducing or eliminating blood flow obstruction to the human heart during and after cardiac valve replacement. For example, a catheter-mounted, intravascular gripping and slitting device may be utilized for aortic valve leaflet modification, to prevent obstruction of coronary arteries during or following an aortic valve replacement procedure.

### BACKGROUND

One common type of valve disease is aortic valve stenosis, in which the valve between the aorta and the heart's left ventricle is narrowed and doesn't fully open. This reduces blood flow from the heart to the aorta and thus to the rest of the body. With the advent of minimally invasive procedures, valve replacement may currently be the most common therapy for aortic valve stenosis. More particularly, transvenous/transcatheter aortic valve replacement (TAVR) is becoming a more common valve replacement procedure, as technology and doctor skillsets improve. This minimally invasive approach to valve replacement is an alternative to open heart surgical aortic valve replacement (SAVR). In a TAVR procedure, the damaged valve, whether the valve is a natural aortic valve or a previous SAVR or TAVR valve, is left in place. These valves may have anatomical abnormalities, calcification, or infection. TAVR has been approved for low-risk patients, who in general are of younger age and live longer. Recent studies have shown that the life of a TAVR valve will only be on average 8 years, so there will be an increase in replacement TAVR procedures. Old TAVR leaflets may be fibrosed, calcified, or thickened during the life of the TAVR valve, creating a barrier to the replacement TAVR valve. The old leaflets may also pose a more serious risk of coronary obstruction than native valves.

Many structural heart procedures, including TAVR and other valve replacement procedures, benefit from modification of the leaflets of native and/or implanted valves. For example, aortic valve replacement can cause the coronary vessels to become occluded when the leaflets of the native valve (or in the case of a valve-in-valve implant, the implanted valve leaflet), are pressed up against the side wall of the aortic valve annular region. Similarly, in a mitral valve replacement, the left ventricular out flow tract (LVOT) can become narrowed by the leaflets of the native valve or previously implanted valve. Another issue can be access to the coronary arteries after implantation of a prosthetic aortic valve. The leaflet of the native or implanted valve can prevent easy access for a guidewire to cross from the coronary sinus area into the ostia of the coronary arteries. Thus, a need exists for improved intracoronary methods to address the foregoing and other concerns.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Disclosed herein is a leaflet gripping and slitting device, with associated systems and methods. The device includes a gripping capability, as well as a heated wire capable of slitting the tissue of the leaflet. This leaflet gripping and slitting device enables a clinician to immobilize the leaflet and then slit it in the proper location, while preventing damage to nearby structures. The grasping apparatus not only controls the position of the leaflet during slitting, but also guides the cutting wire along a straight path, and prevents the cutting wire from cutting into tissue other than the leaflet. The leaflet gripping and slitting device includes gripper that is delivered endovascularly into the heart, and is configured to clamp onto a valve leaflet of interest. Once the leaflet is gripped, a wire heated by radio frequency/electrical energy is advanced along a track or guide in the jaws of the gripper, creating a slit in the valve leaflet from the center of the annular space towards the root of the valve leaflet. The gripper's track or guide prevents the energized wire from cutting too far and/or damaging adjacent structures of the heart.

The leaflet gripping and slitting device has particular but not exclusive utility for modifying heart valve leaflets during intracardiac valve replacements. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a gripping and slitting device, which includes a catheter configured to be positioned within a body lumen of a patient, and a gripping assembly coupled to the catheter. The gripping assembly may include: a first jaw, a second jaw, a hinge mechanism coupled to the first jaw and the second jaw and configured to rotate the first jaw relative to the second jaw between an open state and a closed state. The gripping and slitting device also includes a cutting assembly coupled to the gripping assembly including: at least one conductor, and a cutting element configured to be energized by the at least one conductor. Other embodiments of this aspect include corresponding computer systems, apparatus, and computer programs recorded on one or more computer storage devices, each configured to perform the actions of the methods.

Implementations may include one or more of the following features. In some aspects, the gripping and slitting device may include a guide track disposed within at least one of the first jaw or the second jaw, where the cutting assembly is configured to move along the guide track between a proximal portion and a distal portion of the gripping assembly. In some aspects, the first jaw is fixedly attached to the catheter, and the second jaw is rotatable relative to the first jaw via the hinge mechanism. In some aspects, the cutting assembly is fixed to at least one of the first jaw or the second jaw. In some aspects, the cutting element may include a wire configured to be heated by electrical energy carried by the at least one conductor. In some aspects, the wire may include an exposed portion of the at least one conductor. In some aspects, the gripping and slitting device may include a return electrode configured to be in contact with an exterior of the patient's body. In some aspects, the cutting element may include a plasma-generating electrode. In some aspects, the hinge mechanism may include a hinge pin and a pull wire. In some aspects, the body lumen may include an aorta or a heart chamber of the patient, where the gripping assembly is configured to grip a leaflet of a heart valve of the patient, and where the cutting assembly is configured to slit at least a portion of the leaflet. Implementations of the described techniques may include hardware, a method or process, or computer software on a computer-accessible medium.

One general aspect includes a system which includes a power source, an actuator, and a gripping and slitting device that may include: a catheter configured to be advanced to a heart valve of a patient; a gripping assembly coupled to the catheter and may include: a first jaw; a second jaw; a hinge mechanism coupled to the actuator, the first jaw, and the second jaw, where the hinge mechanism is configured to rotate the first jaw relative to the second jaw between an open state and a closed state, where the first jaw and the second jaw are configured to grip a leaflet of a heart valve in the closed state; and a cutting assembly coupled to the gripping assembly and may include: at least one conductor coupled to the power source; and a cutting element configured to slit the leaflet when energized by the at least one conductor. Other embodiments of this aspect include corresponding computer systems, apparatus, and computer programs recorded on one or more computer storage devices, each configured to perform the actions of the methods.

Implementations may include one or more of the following features. In some aspects, the system may include a guide track disposed within at least one of the first jaw or the second jaw, where the cutting assembly is configured to move along the guide track between a proximal portion and a distal portion of the gripping assembly. In some aspects, the first jaw is fixedly attached to the catheter, and the second jaw is rotatable relative to the first jaw via the hinge mechanism. In some aspects, the cutting assembly is fixed to at least one of the first jaw or the second jaw. In some aspects, the cutting element is a wire configured to be heated by electrical energy carried by the at least one conductor. In some aspects, the wire may include an exposed portion of the at least one conductor. In some aspects, the system may include a return electrode configured to be in contact with an exterior of the patient's body. In some aspects, the cutting element may include a plasma-generating electrode. In some aspects, the hinge mechanism may include a hinge pin and a pull wire. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A is a side view of a human heart according to aspects of the present disclosure.

FIG. 1B is a cross-sectional side view of a human heart according to aspects of the present disclosure.

FIG. 2 is a cross-sectional side view of an aortic valve replacement in a human heart 100 according to aspects of the present disclosure.

FIG. 3A is a diagrammatic, cross-sectional top view of an aortic valve replacement of a degenerated natural aortic valve utilizing a TAVR valve, according to aspects of the present disclosure.

FIG. 3B is a diagrammatic cross-sectional top view of a valve-in-valve replacement of a degenerated TAVR valve utilizing a new TAVR valve, according to aspects of the present disclosure.

FIG. 3C is a diagrammatic cross-sectional top view of a valve-in-valve replacement of a degenerated surgical aortic valve replacement (SAVR) valve utilizing a new TAVR valve, according to aspects of the present disclosure.

FIG. 4A is a diagrammatic top view of an aortic valve, according to aspects of the present disclosure.

FIG. 4B is a diagrammatic cross-sectional side view of a TAVR implant in an aortic valve according to aspects of the present disclosure.

FIG. 5 is schematic, diagrammatic view of a system according to aspects of the present disclosure.

FIG. 6A is a diagrammatic cross-sectional side view of an aortic valve according to aspects of the present disclosure.

FIG. 6B is a diagrammatic cross-sectional side views of an aortic valve according to aspects of the present disclosure.

FIG. 7 is a diagrammatic cross-sectional side views of an aortic valve according to aspects of the present disclosure.

FIG. 8 is a schematic, diagrammatic side view of an example gripper or gripping assembly of a leaflet gripping and slitting device, according to aspects of the present disclosure.

FIG. 9 is a schematic, diagrammatic side cross-sectional view of an example cutting wire assembly of a leaflet gripping and slitting device, according to aspects of the present disclosure.

FIG. 10A is a schematic, diagrammatic side view of an example leaflet gripping and slitting device in a closed configuration, according to aspects of the present disclosure.

FIG. 10B is a schematic, diagrammatic side view of an example leaflet gripping and slitting device in a closed configuration, according to aspects of the present disclosure.

FIG. 11 is a schematic, diagrammatic, front cross-sectional view of an example leaflet gripping and slitting device along line 11-11 from FIG. 10, according to aspects of the present disclosure.

FIG. 12 is a schematic, diagrammatic, front cross-sectional view of an example leaflet gripping and slitting device along line 12-12 from FIG. 10, according to aspects of the present disclosure.

FIG. 13 is a schematic, diagrammatic side view of an example gripper of a leaflet gripping and slitting device, according to aspects of the present disclosure.

FIG. 14 is a schematic, diagrammatic side view of an example gripper of a leaflet gripping and slitting device, according to aspects of the present disclosure.

FIG. 15 is a schematic, diagrammatic side cross-sectional view of an example leaflet gripping and slitting device, according to aspects of the present disclosure.

FIG. 16 is a schematic, diagrammatic side cross-sectional view of an example leaflet gripping and slitting device, according to aspects of the present disclosure.

FIG. 17 is a schematic, diagrammatic side cross-sectional view of an example leaflet gripping and slitting device, according to aspects of the present disclosure.

FIG. 18A is a diagrammatic top view of an aortic valve, according to aspects of the present disclosure.

FIG. 18B is a diagrammatic top view of an aortic valve, according to aspects of the present disclosure.

FIG. 19 is schematic, diagrammatic view of a system according to aspects of the present disclosure.

FIG. 20 is a schematic, diagrammatic, front cross-sectional view of an example leaflet gripping and slitting device, according to aspects of the present disclosure.

FIG. 21 is a schematic, diagrammatic, front cross-sectional view of an example leaflet gripping and slitting device, according to aspects of the present disclosure.

FIG. 22 is a schematic, diagrammatic, side cross-sectional view of an example leaflet gripping and slitting device, according to aspects of the present disclosure.

FIG. 23 is a schematic diagram of a processor circuit, according to aspects of the present disclosure.

### DETAILED DESCRIPTION

Disclosed herein is a leaflet gripping and slitting device, with associated systems and methods. During a heart valve replacement or other intracardiac procedure, slitting a heart valve leaflet from the center of the valve annulus towards the root of the leaflet (or vice versa) can allow for blood flow and/or passage of a guidewire. Procedures incorporating such a slitting process may be known as BASILICA or LAMPOON procedures, depending upon which heart valve is being repaired or replaced. This procedure uses a guidewire to pierce the leaflet near the root, and then the guide wire tip is snared, and externalized. Once both ends of the wire are under direct physician control (outside of the patient body), radio frequency energy is applied to the wire, which allows it to “cut” through the leaflet as tension is applied to the ends of the wire. This creates a slit in the leaflet from the root towards the center of the annular space. However, the BASILICA and LAMPOON procedures can be very technically challenging, requiring very advanced wire technique to both pierce the leaflet, especially in the area intended, and also to snare the tip of the wire to externalize the distal end.

A transcatheter edge to edge repair (TEER) procedure, used to treat mitral valve regurgitation, is a procedure in which the leaflets of the mitral valve are “grabbed” by a clip like device, and then drawn together as the device is deployed.

This leaflet gripping and slitting device enables a clinician to immobilize the leaflet and then slit it in the proper location, while preventing damage to nearby structures. The leaflet gripping and slitting device includes an apparatus to mechanically grab and control a heart valve leaflet during radio frequency (RF, e.g., heated-wire) slitting of said leaflet. The grasping apparatus not only controls the position of the leaflet during slitting, but also guides the radio frequency cutting wire along a straight path, and prevents the cutting wire from cutting into tissue other than the leaflet.

The leaflet gripping and slitting device includes a gripper device that is delivered endovascularly into the heart, and is configured to clamp onto a valve leaflet of interest. Once the leaflet is gripped, an RF-heated wire is advanced along a track or guide in the jaws of the gripper, creating a slit in the valve leaflet from the center of the annular space towards the root of the valve leaflet. The gripper's track or guide prevents the energized wire from cutting too far and/or damaging adjacent structures of the heart. Although only a single slit is normally required, the device can also be used to add additional slits to either the same leaflet, adjacent leaflets, or leaflets of another valve entirely.

In an example, a flexible elongate member (e.g., a catheter) is equipped with a central lumen, a pull wire, and a set of jaws at the distal end, similar to an alligator clip or gripper type catheter. In addition to the pull wire, the central lumen allows for the passage of a shaped wire to the leaflet gripping and slitting device. The jaws incorporate a slot or slots that run along the axis of the catheter, but stop before the distal ends of the jaws. The shaped wire, when pushed from the proximal end of the catheter, traverses along the groves in the jaws, and is stopped when the wire reaches the ends of the groove(s). The pull wire, attached to one or both of the jaws, allows the user to open and close the jaws from the proximal end of the catheter.

In use, the catheter is introduced into the vasculature of the patient and directed to the valve leaflet of interest. Once there, the user opens the jaws and gains control of the leaflet of interest. If the user is satisfied with the amount of leaflet entrapped by the jaws, as well as the location being grasped, radio frequency energy from a standard electrocautery source is applied to the shaped wire, which is advanced within the lumen of the catheter. As the wire advances, it cuts the tissue entrapped by the jaws until it reaches the distal end of the groove(s) in the jaw(s). The amount of clearance between the outside diameter of the wire and the width of the groove(s) in the jaw(s) allows for the wire to maneuver around calcium or other obstructions to at least some extent.

The present disclosure aids substantially in intracardiac procedures such as heard valve replacement, by improving a clinician's ability to cut (e.g., bisect) a valve leaflet with precision, and with minimal risk of damage to neighboring tissues. Implemented on an intracardiac catheter in association with an intracardiac catheter system, the leaflet gripping and slitting device disclosed herein provides practical, precise surgical capabilities in an intracardiac environment. This improved leaflet modification technique transforms a complex, potentially imprecise procedure requiring high levels of skill and training into a repeatably precise leaflet slitting technique, without the normally routine need to achieve this precision through open-heart surgical interventions. This unconventional approach improves the functioning of the intracardiac catheter system, by allowing a greater range of procedures to be reliably performed using intracardiac techniques.

Control of the leaflet gripping and slitting device may be implemented at least partially through ultrasound or X-ray imaging under the control of a processor and viewable on a display, and operated by a control process executing on a processor that accepts user inputs from a keyboard, mouse, or touchscreen interface. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

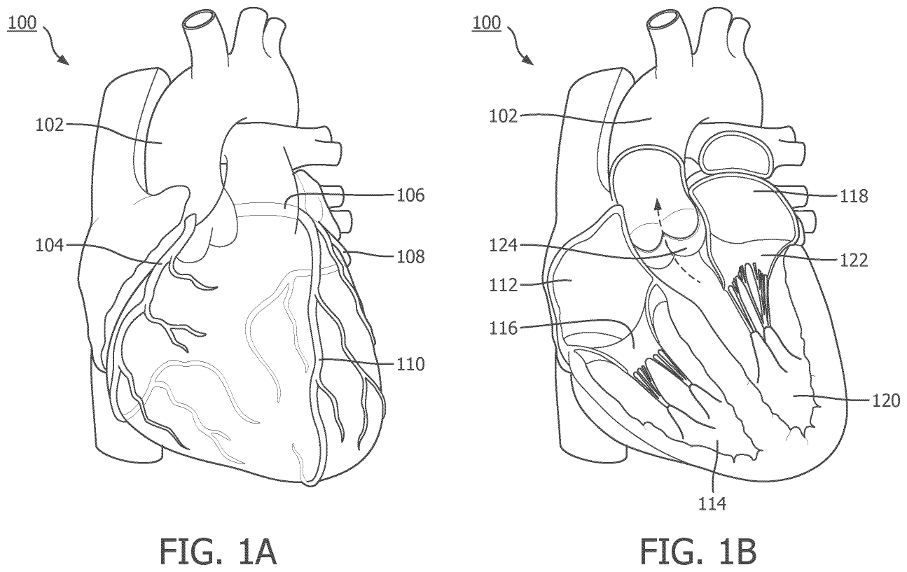

FIG. 1A is a side view of a human heart 100 according to aspects of the present disclosure. Visible are an aorta 102 from which stems a right coronary artery 104 and a left main coronary artery 106. The left main coronary artery 106 branches into a left circumflex coronary artery 108 and a left anterior descending coronary artery 110. The right coronary artery 104, the left main coronary artery 106, the left circumflex coronary artery 108, and a left anterior descending coronary artery 110 are the arteries that provide oxygen-rich blood to muscles of the human heart 100.

FIG. 1B is a cross-sectional side view of a human heart 100 according to aspects of the present disclosure. Visible are a right atrium 112 and a right ventricle 114. In that regard, oxygen-poor blood enters the human heart 100 in the right atrium 112 and travels to the right ventricle 114 through the tricuspid valve 116. The oxygen-poor blood leaves the right ventricle 114 and travels to the lungs. Also visible are a left atrium 118 and a left ventricle 120. In that regard, oxygen-rich blood is received from the lungs in the left atrium 118 and travels to the left ventricle 120 through the mitral valve 122. The oxygen-rich blood leaves the left ventricle 120 and goes out to the body through the aorta 102 via an aortic valve 124.

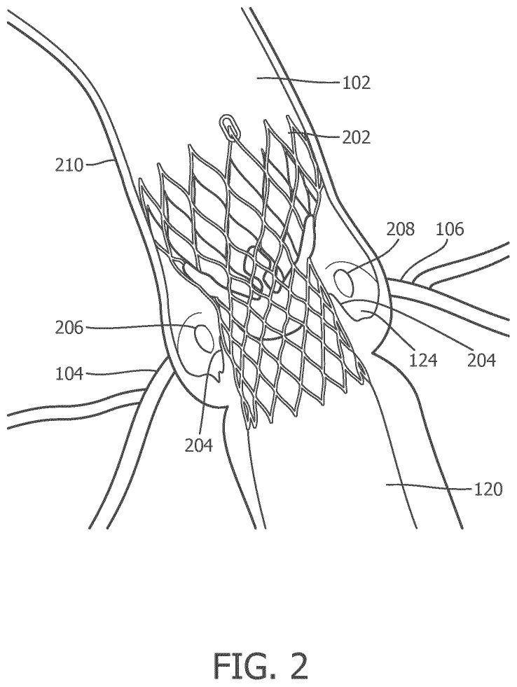

FIG. 2 is a cross-sectional side view of an aortic valve 124 replacement in a human heart 100 according to aspects of the present disclosure. In some aspects, e.g., when aortic valve stenosis has occurred to the aortic valve 124 that keeps blood flowing in the correct direction from the left ventricle 120 to the aorta 102, a transvenous/transcatheter aortic valve repair (TAVR) procedure may be performed to replace the natural aortic valve 124 with a replacement aortic valve 202. In some instances, portions of one or more leaflets 204 of the natural aortic valve 124 may be resected so that openings 206 and 208 to the right coronary artery 104 and the left main coronary artery 106, respectively, so that, when the one or more leaflets 204 are pressed against the natural heart wall 210, the openings 206 and 208 remain unobstructed so that oxygen-rich blood may flow to the muscles of the human heart. The natural heart wall 210 may be a natural aorta wall, a natural heart chamber wall, or a natural aortic valve wall.

The TAVR procedure is shown here for exemplary purposes only; it is understood that other heart valves and heart valve replacement procedure types may benefit from leaflet modification and thus fall within the scope of the present disclosure.

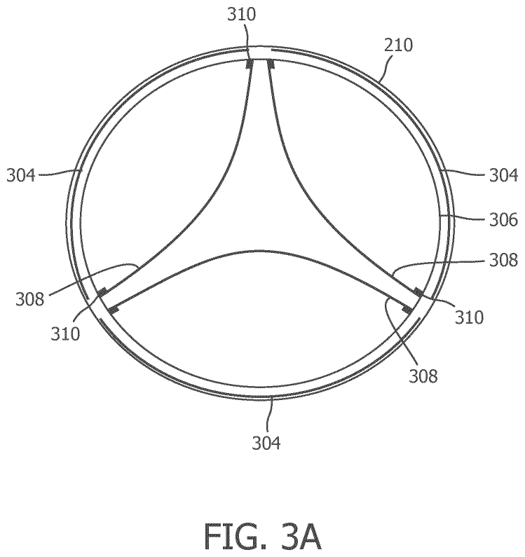

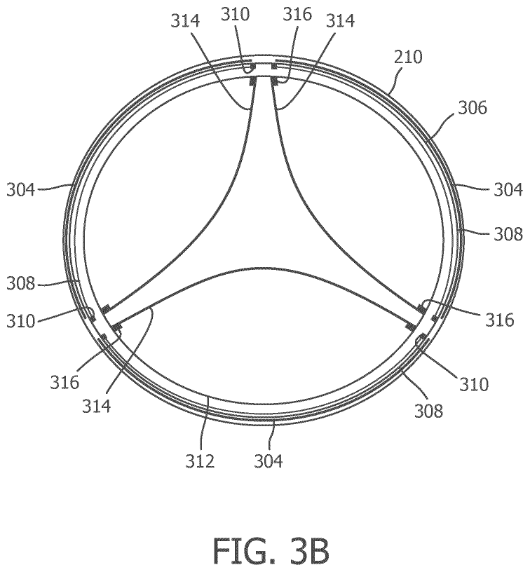

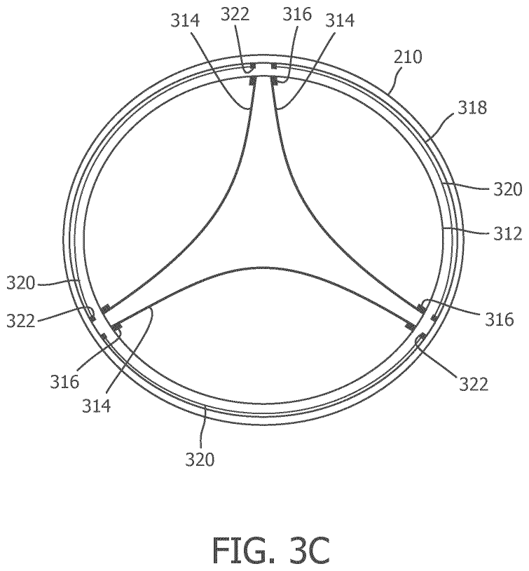

FIG. 3A is a diagrammatic, cross-sectional top view of an aortic valve replacement of a degenerated natural aortic valve utilizing a TAVR valve, according to aspects of the present disclosure. Visible are the natural heart wall 210, the natural aortic valve leaflets 304, the TAVR valve wall 306, the TAVR valve leaflets 308, and the TAVR commissural tabs 310. The TAVR valve leaflets 308 may for example be constructed from bovine animal tissue coupled to the wire frame of the TAVR valve wall 306 via the commissural tabs 310. When inserted, the TAVR valve pushes the remaining unresected portions of the natural aortic valve leaflets 304 against the natural heart wall 210 such that the natural aortic valve leaflets 304 are pinned and/or secured between an outside of the TAVR valve wall 306 and the natural heart wall 210.

FIG. 3B is a diagrammatic cross-sectional top view of a valve-in-valve replacement of a degenerated TAVR valve utilizing a new TAVR valve, according to aspects of the present disclosure. Visible are the natural heart 210, the natural aortic valve leaflets 304, the degenerated TAVR valve wall 306, the degenerated TAVR valve leaflets 308, the degenerated TAVR commissural tabs 310, the new TAVR valve wall 312, the new TAVR valve leaflets 314, and the new TAVR commissural tabs 316. The new TAVR valve leaflets 314 are constructed from bovine animal tissue are coupled to the wire frame of the new TAVR valve wall 312 via the commissural tabs 316. When inserted, the TAVR valve pushes the remaining unresected portions of the degenerated TAVR valve leaflets 308 against an inside of the degenerated TAVR valve wall 306 such that the degenerated TAVR valve leaflets 308 are pinned and/or secured between an outside of the new TAVR valve wall 312 and the inside wall of the degenerated TAVR valve wall 306.

FIG. 3C is a diagrammatic cross-sectional top view of a valve-in-valve replacement of a degenerated surgical aortic valve replacement (SAVR) valve utilizing a new TAVR valve, according to aspects of the present disclosure. Visible are the natural heart wall 210, a degenerated SAVR valve wall 318, the degenerated SAVR valve leaflets 320, the degenerated SAVR commissural tabs 322, the new TAVR valve wall 312, the new TAVR valve leaflets 314, and the new TAVR commissural tabs 316. The new TAVR valve leaflets 314 are constructed from bovine animal tissue and are coupled to the wire frame of the new TAVR valve wall 312 via the commissural tabs 316. When inserted, the TAVR valve pushes the remaining unresected portions of the degenerated SAVR valve leaflets 320 against an inside of the degenerated SAVR valve wall 318 such that the degenerated TAVR valve leaflets 320 are pinned and/or secured between an outside of the new TAVR valve wall 312 and the inside wall of the degenerated SAVR valve wall 318.

The natural valve, TAVR valve, and/or SAVR valve can be degenerated (e.g., not fully or correctly functioning) as a result of various causes. For example, the natural valve, TAVR valve, and/or SAVR valve can be degenerated because it is stenosed, because it is regurgitant, etc.

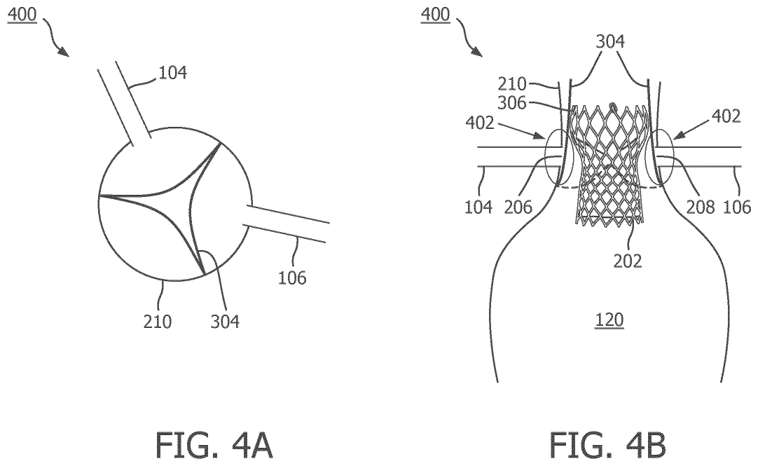

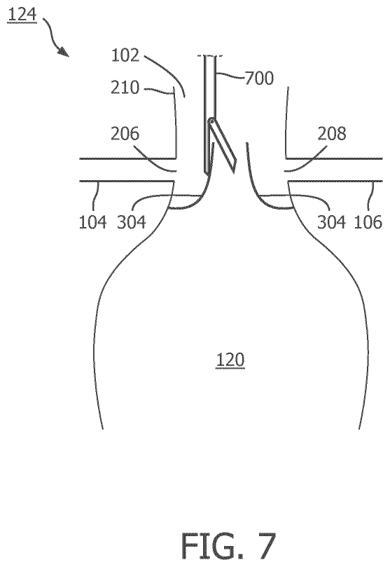

FIG. 4A is a diagrammatic top view of an aortic valve 400, according to aspects of the present disclosure. Visible are the natural heart wall 210, the aortic valve leaflets 304, the right coronary artery 104, and the left main coronary artery 106. During and after valve replacement procedures, the leaflets 304 can obstruct the arteries 104, 106, as described below in FIG. 4B. This can be alleviated by resecting one or more leaflets 304, as shown below in FIGS. 7 and 15-18B.

FIG. 4B is a diagrammatic cross-sectional side view of a TAVR implant in an aortic valve 400 according to aspects of the present disclosure. Visible are the natural heart wall 210, the aortic valve leaflets 304, the right coronary artery 104, and the left main coronary artery 106. In some aspects, when a replacement aortic valve 202 is implanted and the aortic valve leaflets 304 are not resected or insufficiently resected, the aortic valve leaflets 304, when pinned and/or secured between an outside of the TAVR valve wall 306 and the natural heart wall 210, obstruct the openings 206 and 208 to the right coronary artery 104 and the left main coronary artery 106, respectively, as indicated by areas 402.

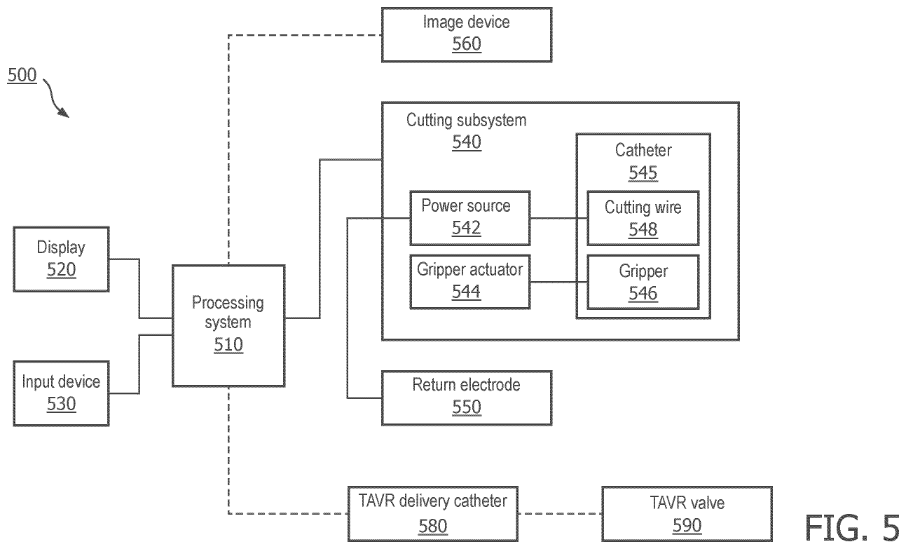

FIG. 5 is schematic, diagrammatic view of a system 500 according to aspects of the present disclosure. The system 500 may be configured to evaluate (e.g., assess), display, and/or control (e.g., modify) one or more aspects of a cardiac valve replacement. For instance, the system 500 may be utilized to monitor and/or control one or more portions of a cardiac valve replacement process while reducing and/or eliminating aortic regurgitation, as described in greater detail below. In this regard, the system 500 may be used to assess coronary vessels and/or heart tissue (e.g., the myocardium). As illustrated, the system 500 may include a processing system 510 in communication with a display device 520 (e.g., an electronic display or monitor), an input device 530 (e.g., a user input device, such as a keyboard, mouse, joystick, microphone, and/or other controller or input device), a cutting subsystem 540 and/or an imaging device 560. As illustrated, the system 500 may further include a TAVR delivery catheter 580 and a TAVR valve 590, which may be completely mechanical or connected to processing system 510 in order to provide intraluminal data to processing system 510. It is understood that other types of valves and valve delivery systems may be used instead of or in addition to TAVR.

The cutting system 540 includes a power source 542 coupled to a cutting wire 548, and a gripper actuator 544 coupled to a gripper 546. The cutting wire 548 and gripper 546 are delivered by a catheter 545 to the site of the leaflet of interest. Once properly located, the gripper 546 may be extended from a distal end of the catheter 545 to grip the leaflet, and the cutting wire 548 may be deployed within the gripper to slit the leaflet as shown below. In some cases, the cutting subsystem 540 may require a return electrode 550 in contact with the outside of the patient's body. In other instances, the cutting wire may be a loop that includes both a source and return.

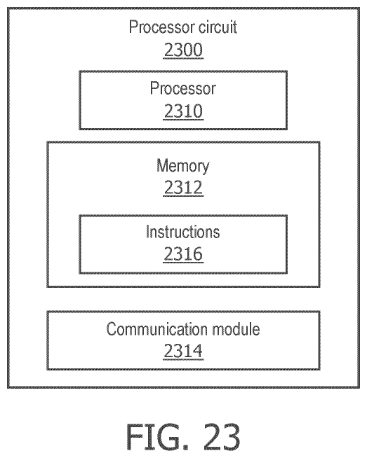

The processing system 510 is generally representative of any device suitable for performing the processing and analysis techniques disclosed herein. In some aspects, the processing system 510 includes a processor circuit, such as the processor circuit 2300 of FIG. 23. In some aspects, the processing system 510 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processing system 510 (e.g., computing device) using corresponding instructions stored on or in a non-transitory computer readable medium accessible by the computing device. In some instances, the processing system 510 is a console device. Further, it is understood that in some instances the processing system 510 includes one or a plurality of computing devices, such as computers, with one or a plurality of processor circuits. In this regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure. In some instances, the system 500 omits the processing system 510 and/or other components of the system 500 (e.g., input device 530). For example, the system 500 can include the cutting subsystem 540, without the processing system 510. The gripper actuator 544 and/or the 545 can be manually activated/controlled by a user. The power source 542 can be manually activated/controlled by a user, using a user input on the power source 542 itself (e.g., a switch for on/off, a knob for power/current/voltage level, etc.).

The system 500 is configured such that when the gripper 546 is positioned within the heart, images captured by the imaging system 560 can show the location and open-closed state of the gripper, and also potentially anatomical features such as heart valve leaflets. The cutting wire 548 may also technically be contained within the images, but may be difficult to resolve, as the cutting wire 548 is located within the gripper 546.

It is noted that block diagrams are provided herein for exemplary purposes; a person of ordinary skill in the art will recognize myriad variations that nonetheless fall within the scope of the present disclosure. For example, block diagrams may show a particular arrangement of components, subcomponents, modules, units, etc. It is understood that some embodiments of the systems disclosed herein may include additional components, that some components shown may be absent from some embodiments, and that the arrangement of components may be different than shown, while still performing the methods described herein.

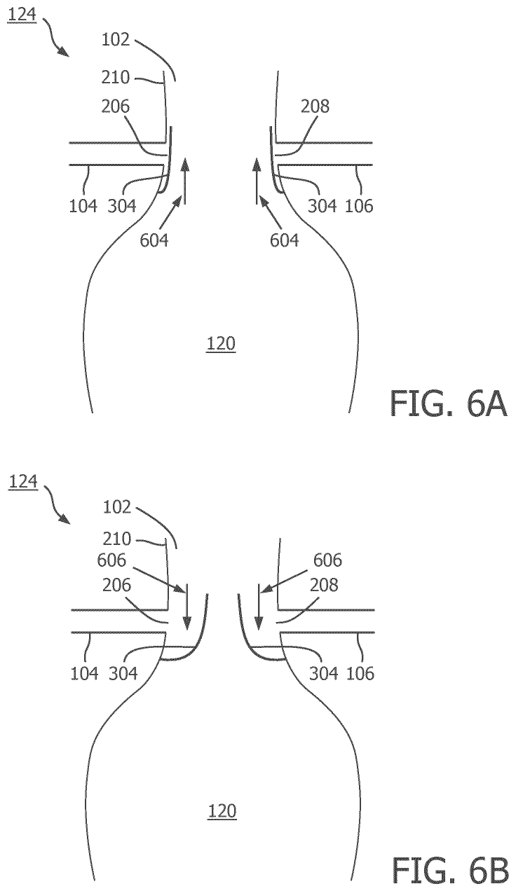

FIG. 6A is a diagrammatic cross-sectional side view of an aortic valve 124 according to aspects of the present disclosure. Visible are the natural heart wall 210, the aortic valve leaflets 304, the right coronary artery 104, and the left main coronary artery 106. In the aspects of FIG. 6A, during systole (indicated by directional arrows 604), the aortic valve leaflets 304 open and allow blood to flow from the left ventricle 120 to the aorta 102 indicated by directional arrows 604.

FIG. 6B is a diagrammatic cross-sectional side views of an aortic valve 124 according to aspects of the present disclosure. Visible are the natural heart wall 210, the aortic valve leaflets 304, the right coronary artery 104, and the left main coronary artery 106. In the aspects of FIG. 6B, during diastole (indicated by directional arrows 606), the aortic valve leaflets 304 may be pushed by the flow of blood from the aorta 102 toward the left ventricle 120 such that the aortic valve leaflets 304 close to prevent mitral regurgitation.

FIG. 7 is a diagrammatic cross-sectional side views of an aortic valve 124 according to aspects of the present disclosure. In the example shown in FIG. 7, a leaflet gripping and slitting device 700 is being positioned onto one of the leaflets 304, in order to grip and slit the leaflet as described below. The leaflet gripping and slitting device 700 advantageously enables the replacement of an aortic valve 124 in a non-obstructive way, by resecting old valve leaflets 304 and preparing the implant site for a cleaner valve deployment and operation. For example, resecting portions of the leaflets 304 of the degenerated aortic valve can allow the replacement valve to be expanded without the current leaflets 304 covering the openings to the coronary arteries 104, 106, thereby allowing oxygenated blood to flow to the muscles of the heart.

It is noted that the orientation of the leaflet gripping and slitting device 700 in use can be flipped relative to what is what is shown in FIG. 7. In FIG. 7, the moveable jaw is on the inside of the annulus and the moveable jaw is between the leaflet and the wall of the aorta. In other instances, the fixed jaw could be on the inside of the annulus and the moveable jaw could be between the leaflet and the wall of the aorta. Although FIG. 7 illustrates the leaflet gripping and slitting device 700 being used on the aortic valve 124, it is also noted that that the device can be used to resect a leaflet of any heart valve (e.g., an aortic valve, mitral valve, pulmonary valve, or tricuspid valve). Furthermore, the leaflet gripping and slitting device 700 could be in the heart (e.g., a heart chamber) or a great vessel (e.g., the aorta) while it is gripping and slitting the leaflet.

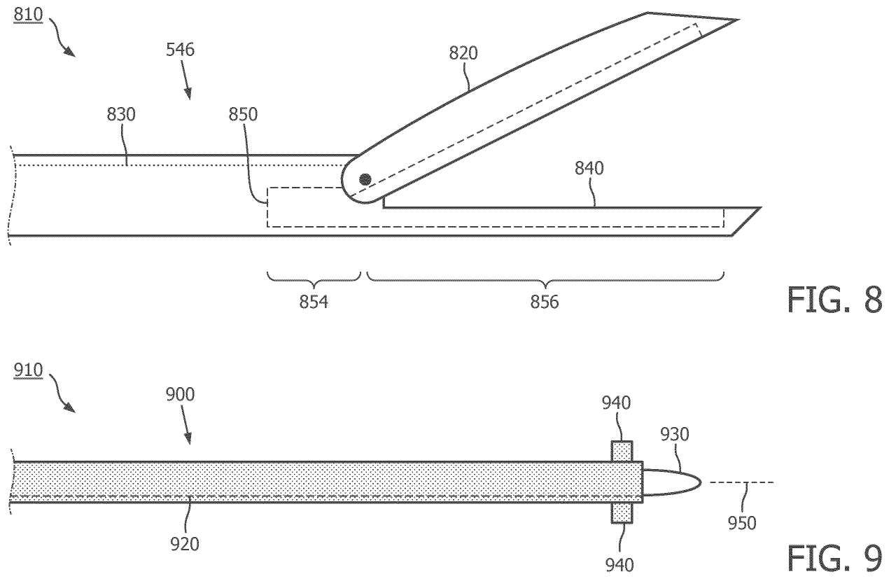

FIG. 8 is a schematic, diagrammatic side view of an example gripper or gripping assembly 546 of a leaflet gripping and slitting device, according to aspects of the present disclosure. The gripper 546 includes a body 810 (which may for example be a catheter), a movable upper jaw 820 actuated by a pull wire 830, a fixed lower jaw 840, and a guide track or channel 850 (or plurality of tracks or channels). When actuated by the pull wire 830, the movable upper jaw 820 rotates around a hinge pin 860. Thus, the hinge pin and pull wire may collectively be referred to as a hinge mechanism, which is coupled to the upper jaw 820 and the lower jaw 840, and is coupled to the gripper actuator (see FIG. 5) via the pull wire 830. In the example shown in FIG. 8, since the gripper 546 is shown in the open configuration, the track or channel 850 includes an enclosed proximal portion 854 and an open distal portion 856. It is noted that the fixed jaw and moveable jaw can be switched, e.g., the tissue gripping and slitting device can have a fixed upper jaw and moveable lower jaw.

FIG. 9 is a schematic, diagrammatic side cross-sectional view of an example cutting wire assembly 900 of a leaflet gripping and slitting device, according to aspects of the present disclosure. The cutting wire assembly 900 includes a body 910 (which may for example be a catheter), a conductor 920 coupled to a cutting wire loop 930, and guide pegs 940 configured to slide within the guide track(s) or channel(s) 850 of FIG. 8. The longitudinal axis 950 of the cutting wire assembly 900 is also shown. The conductor 920 carries electrical energy (voltage and/or current, e.g., alternating at radio frequencies (RF)), thereby energizing and heating the cutting wire loop 930 such that it can slit the tissue of the leaflet. In some aspects, wire 930 can be an exposed portion of conductor 920 (e.g., wire 930 and conductor 920 may be different portions of the same component). In other aspects, wire 930 can be coupled to conductor 920 (e.g., wire 930 may be a separate component that is mechanically and electrically coupled to the conductor 920).

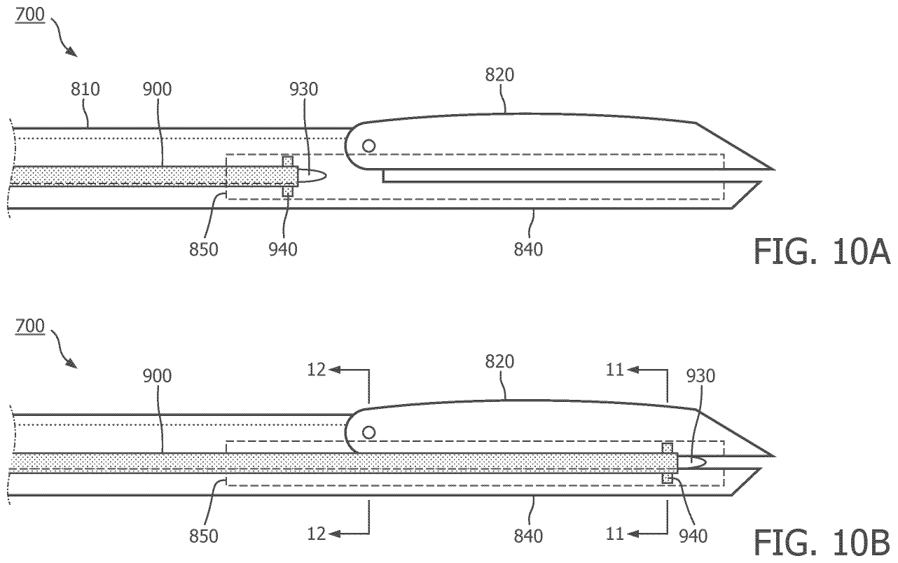

FIG. 10A is a schematic, diagrammatic side view of an example leaflet gripping and slitting device 700 in a closed configuration, according to aspects of the present disclosure. Visible are the body 810, upper jaw 820, lower jaw 840, and cutting wire assembly 900. The guide pegs 940 slide within the guide track 850. In the example shown in FIG. 10A, the cutting wire assembly 900 is in a retracted position, such that the cutting wire loop 930 does not extend outside the body 810.

FIG. 10B is a schematic, diagrammatic side view of an example leaflet gripping and slitting device 700 in a closed configuration, according to aspects of the present disclosure. Visible are the body 810, upper jaw 820, lower jaw 840, and cutting wire assembly 900. The guide pegs 940 slide within the guide track 850. In the example shown in FIG. 10B, the cutting wire assembly 900 is in an extended position, such that the cutting wire loop 930 extends outside the body 810, along the guide track 850 between the upper jaw 820 and lower jaw 840. This extended configuration allows slitting of a heart valve leaflet that is gripped between the upper jaw 820 and lower jaw 840, as described below.

It is noted that parasitic leakage of the current from the RF generator through the device (e.g., into the blood or other tissues nearby that are not intended to be cut) may be undesirable, and that preventing such parasitic leakage currents may be important. It is therefore noted that the design of the leaflet gripping and slitting device 700 aids in the exclusion of conductive blood current leakage. The grasper concept intrinsically provides shielding or protection of the cutting action from fluids in the body when the upper jaw 820 and lower jaw 840 are closed together on the leaflet. This also makes it easier to, for example, flush that shielded space (e.g., the guide track 850) with either a less conductive fluid or non-conductive fluid via a lumen that connects with that space for that purpose. The fact that the grasper jaws 820, 840 would protect that flushing fluid from being washed away by the flow of blood is a distinct advantage of the disclosed design.

It is further noted that the exemplary leaflet gripping and slitting device 700 of FIG. 10B is a monopolar cutting design, where the current passes from the generator, through the device, into tissue in contact with the device, through the body, and into a return electrode, as shown for example in FIG. 5. However, bi-polar designs, wherein at least one of the two jaws provides a return path or ground path, also fall within the scope of the present disclosure.

FIG. 11 is a schematic, diagrammatic, front cross-sectional view of an example leaflet gripping and slitting device 700 along line 11-11 from FIG. 10, according to aspects of the present disclosure. Visible are the upper jaw 820, lower jaw 840, guide track 850, guide pegs 940, cutting wire assembly body 900, and conductor 920. The guide pegs 940 are confined within the guide track 850 such that the cutting wire assembly is free to move only along its longitudinal axis, and is prevented from rotating, bending, or translating along any radial axis. This single-degree-of-freedom (1DOF) motion can help to ensure a clean cut of the grasped leaflet, with minimal risk of damage to other tissues.

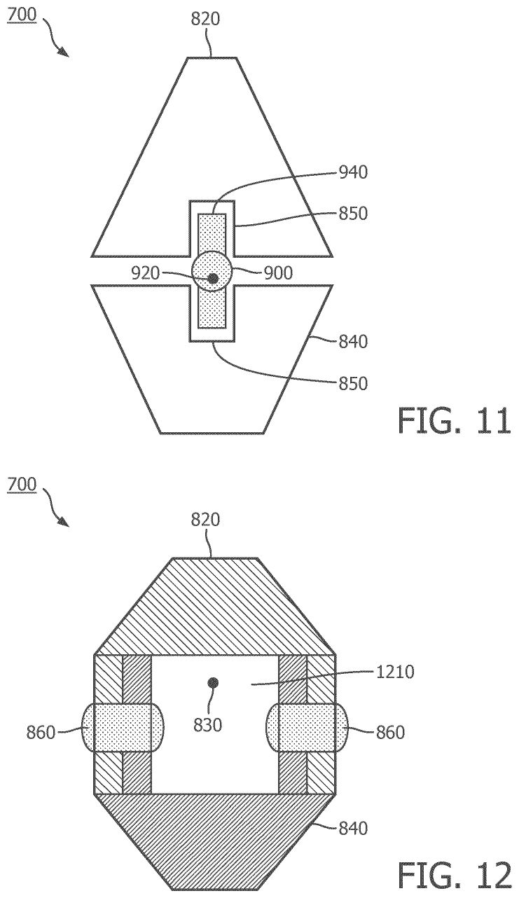

FIG. 12 is a schematic, diagrammatic, front cross-sectional view of an example leaflet gripping and slitting device 700 along line 12-12 from FIG. 10, according to aspects of the present disclosure. Visible are the upper jaw 820, lower jaw 840, hinge pins 860, and a central lumen 1210 through which the pull wire 830 can pass. As shown, the hinge pins 860 are rotatably coupled to both the movable upper jaw 820 and the fixed lower jaw 840, thus permitting the gripper to open and close as the pull wire 830 is actuated. In the example shown in FIG. 12, the hinge feature of the gripper includes two separate hinge pins 860. It is understood that there could instead be a single hinge pin 860 spanning the width of the gripper, although this could limit the possible placement areas for the pull wire 830. It is understood that the additional features may be present, such as springs, serrations, a push rod, a guidewire, a pressure sensor, temperature sensor, or jaw angle sensor, and otherwise, as would occur to a person of ordinary skill in the art to support the functioning of the leaflet gripping and slitting device 700 as described herein.

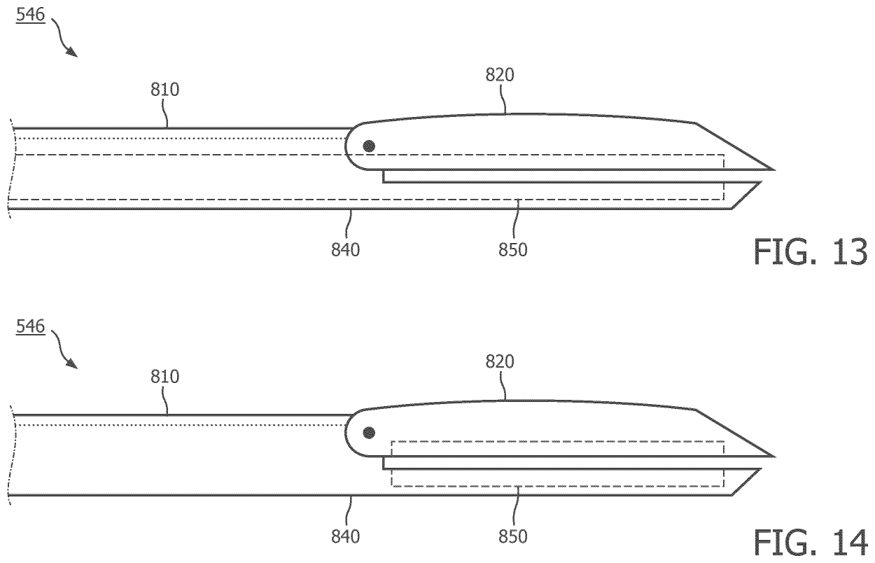

FIG. 13 is a schematic, diagrammatic side view of an example gripper 546 of a leaflet gripping and slitting device, according to aspects of the present disclosure. Visible are the movable upper jaw 820, fixed lower jaw 840, body 810, and guide track 850. In the example shown in FIG. 13, the guide track 850 extends into the body 810, well proximal of the jaws 820, 840, and may for example extend all the way to the proximal end of the body, or may terminate at any location between the jaws 820, 840 and the proximal end of the body 810.

FIG. 14 is a schematic, diagrammatic side view of an example gripper 546 of a leaflet gripping and slitting device, according to aspects of the present disclosure. Visible are the movable upper jaw 820, fixed lower jaw 840, body 810, and guide track 850. In the example shown in FIG. 14, the guide track 850 does not extend into the body 810 at all, but terminates along the length of the jaws 820, 840. Other configurations, different than those shown herein but performing the same or similar functions, fall within the scope of the present disclosure.

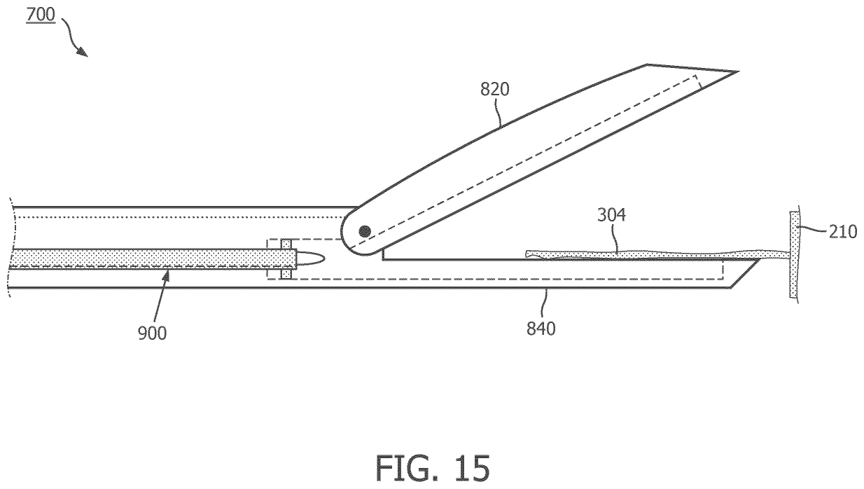

FIG. 15 is a schematic, diagrammatic side cross-sectional view of an example leaflet gripping and slitting device 700 in the open state, according to aspects of the present disclosure. In the example shown in FIG. 15, the upper jaw 820 and lower jaw 840 are being positioned to grip a leaflet 304 extending from the heart wall 210. The cutting wire assembly 900 is shown in a retracted position.

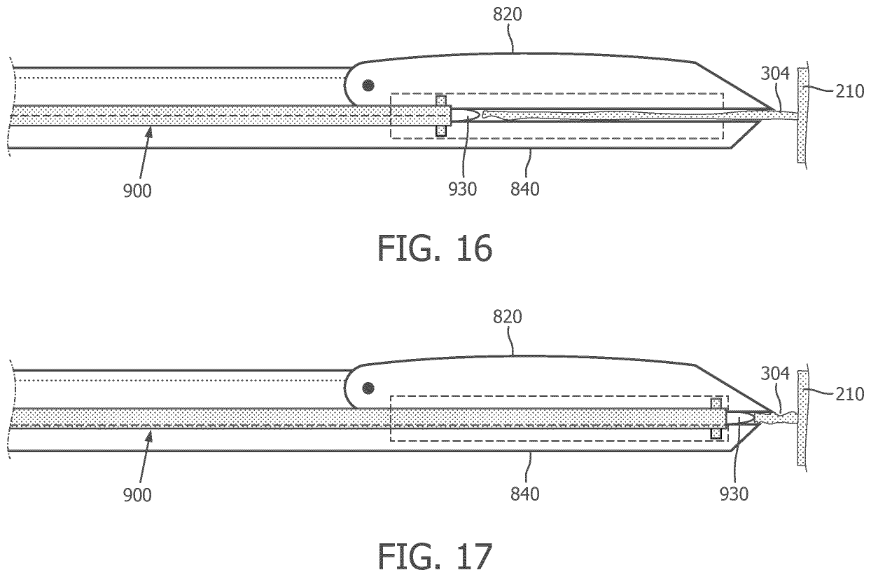

FIG. 16 is a schematic, diagrammatic side cross-sectional view of an example leaflet gripping and slitting device 700 in the closed state, according to aspects of the present disclosure. In the example shown in FIG. 15, the upper jaw 820 and lower jaw 840 have been closed together to grip a leaflet 304 extending from the heart wall 210, but are not in contact with the heart wall 210. Such positioning may allow the leaflet 304 to be slit without damage to the heart wall 210. The cutting wire assembly 900 is shown in a partially extended position, such that the cutting wire 930 is nearly in contact with the leaflet 304.

FIG. 17 is a schematic, diagrammatic side cross-sectional view of an example leaflet gripping and slitting device 700, according to aspects of the present disclosure. In the example shown in FIG. 15, the upper jaw 820 and lower jaw 840 have been closed together to grip a leaflet 304 extending from the heart wall 210. The cutting wire assembly 900 is shown in a fully extended position, such that the cutting wire 930 has been pushed through the majority of the leaflet 304, but does not come in contact with the heart wall 210. Thus, the leaflet gripping and slitting device 700 has enabled the user (e.g., a heart surgeon) to slit the leaflet 304 without damage to the heart wall 210 or other neighboring tissues.

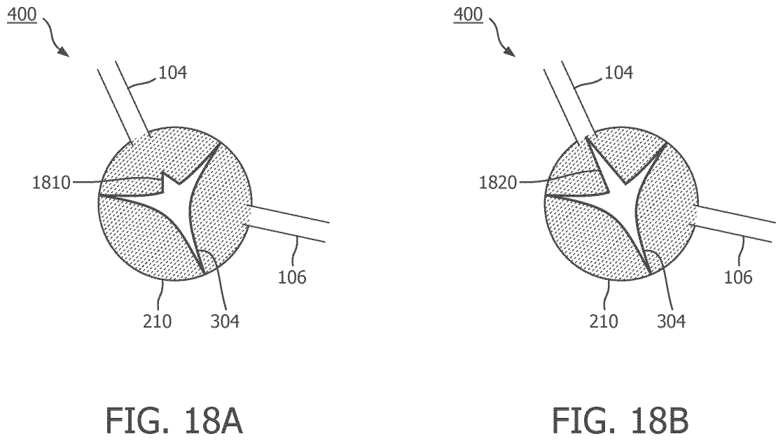

FIG. 18A is a diagrammatic top view of an aortic valve 400, according to aspects of the present disclosure. Visible are the heart wall 210, the aortic valve leaflets 304, the right coronary artery 104, and the left main coronary artery 106. During and after valve replacement procedures, the leaflets 304 can obstruct the arteries 104, 106, as described above. This and other issues can, in certain circumstances, be alleviated by resecting one or more leaflets 304. Thus, one leaflet has been given a partial resection cut 1810 (e.g., by a leaflet gripping and slitting device whose cutting wire is advanced only partway along the length of the leaflet 304).

FIG. 18B is a diagrammatic top view of an aortic valve 400, according to aspects of the present disclosure. Visible are the heart wall 210, the aortic valve leaflets 304, the right coronary artery 104, and the left main coronary artery 106. In the example shown in FIG. 18B, one leaflet has been given a total resection cut 1820 (e.g., by a leaflet gripping and slitting device 700 whose cutting wire is advanced along the length of the leaflet 304 until it is proximate with, but not touching, the heart wall 210).

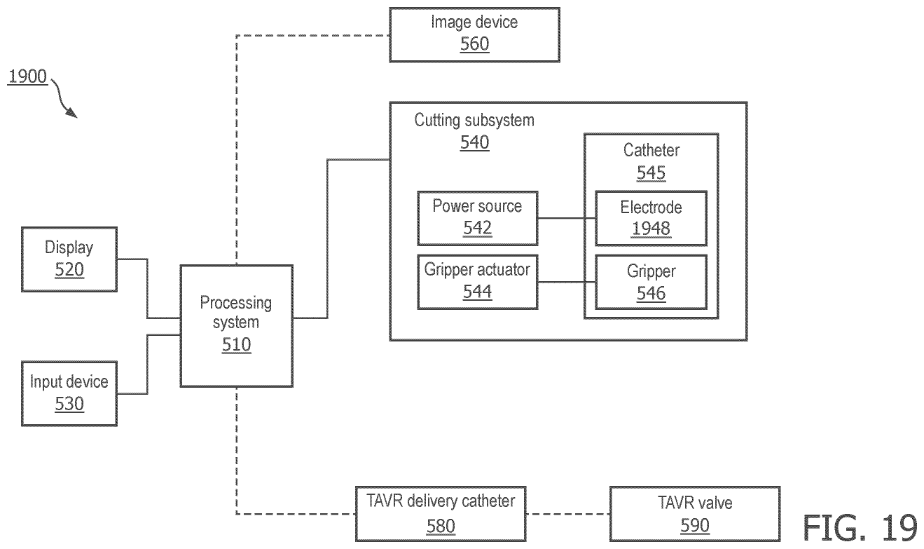

FIG. 19 is schematic, diagrammatic view of a system 1900 according to aspects of the present disclosure. The system 1900 is similar to the system 500 of FIG. 5, except that the cutting wire 548 has been replaced with a plasma-generating electrode 1948, and the return electrode 550 is not present. Rather, the power source 542 forms a complete circuit with the plasma-generating electrode 1948 through the catheter 545. This arrangement allows for another aspect of the leaflet gripping and slitting device, wherein the cutting is not performed by a cutting wire heated with RF energy, but rather by hot plasma generated by the plasma-generating electrode 1948.

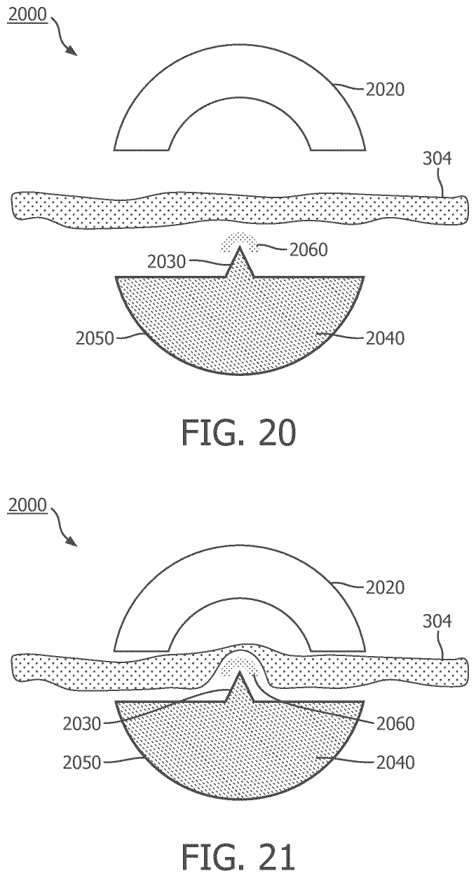

FIG. 20 is a schematic, diagrammatic, front cross-sectional view of an example leaflet gripping and slitting device 2000, according to aspects of the present disclosure. The leaflet gripping and slitting device 2000 includes a movable upper jaw 2020 and a fixed lower jaw 2040, which are positioned over a leaflet 304. The lower jaw 2040 includes an insulating outer layer 2050 and an electrode 2030 that generates plasma 2060. The electrode 2030 may be operated by electrical wires connected to the power source 542 of FIG. 19.

FIG. 21 is a schematic, diagrammatic, front cross-sectional view of an example leaflet gripping and slitting device 2000, according to aspects of the present disclosure.

Visible are the electrode 2030, plasma 2060, and insulating layer 2050, as well as the movable upper jaw 2020 and fixed lower jaw 2040, which have closed onto a leaflet 304. In the example shown in FIG. 21, the plasma 2060 is cutting through the material of the leaflet 304, in a manner similar to an RF-heated cutting wire.

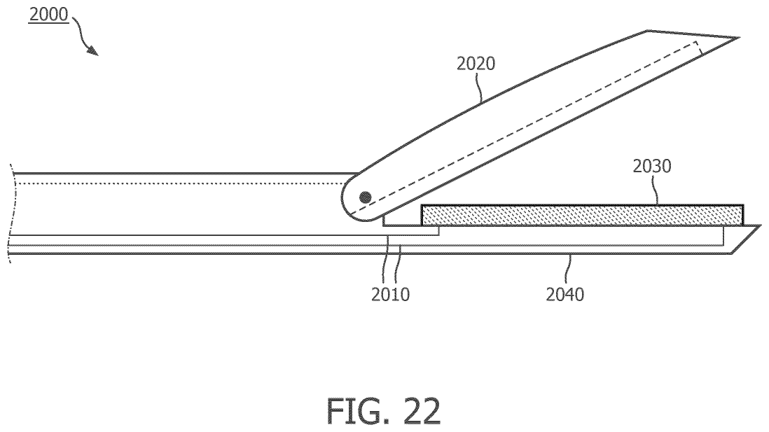

FIG. 22 is a schematic, diagrammatic, side cross-sectional view of an example leaflet gripping and slitting device 2000, according to aspects of the present disclosure. Visible are the upper jaw 2020, lower jaw 2040, and plasma-generating electrode 2030, with control wires 2010 configured to deliver RF energy or electrical energy (voltage and/or current) to energize the electrode 2030. Because the electrode 2030 can be of a size sufficient to cut an entire leaflet at once, the aspect shown in FIGS. 19-22 may not require a movable cutting assembly or a guide track. However, in some related aspects, the electrode 2030 may be mounted on a movable cutting assembly in a manner similar to the RF-heated cutting wire 930 of FIGS. 10A and 10B. In still other aspects, an elongated RF-heated cutting wire 930 may be mounted to the lower jaw in a manner similar to the electrode 2030 shown here in FIG. 22. Thus, in a more general sense, both the plasma-generating electrode 2030 and the RF-heated cutting wire 930 may be referred to as energized cutting elements, and the cutting assembly may be either a movable cutting assembly or a cutting assembly that is fixed to one of the jaws of the gripper. Other types of energized cutting elements, whether fixed relative to the jaws or attached to a movable cutting assembly, also fall within the scope of the present disclosure.

FIG. 23 is a schematic diagram of a processor circuit 2300, according to aspects of the present disclosure. The processor circuit 2300 may be implemented in the processing system 510, the system 500, the system 1900, or other devices or workstations (e.g., third-party workstations, network routers, etc.), or on a cloud processor or other remote processing unit, as necessary to implement the method. As shown, the processor circuit 2300 may include a processor 2310, a memory 2312, and a communication module 2314. These elements may be in direct or indirect communication with each other, for example via one or more buses.

The processor 2310 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 2310 may also comprise another hardware device, a firmware device, or any combination thereof configured to perform the operations described herein. The processor 2310 may also be implemented as a combination of computing devices, e.g., a combination of a DSP and a microprocessor, a plurality of microprocessors, one or more microprocessors in conjunction with a DSP core, or any other such configuration.

The memory 2312 may include a cache memory (e.g., a cache memory of the processor 2310), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 2312 includes a non-transitory computer-readable medium. The memory 2312 may store instructions 2316. The instructions 2316 may include instructions that, when executed by the processor 2310, cause the processor 2310 to perform the operations described herein. Instructions 2316 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 2314 can include any electronic circuitry and/or logic circuitry to facilitate direct or indirect communication of data between the processor circuit 2300, and other processors or devices. In that regard, the communication module 2314 can be an input/output (I/O) device. In some instances, the communication module 2314 facilitates direct or indirect communication between various elements of the processor circuit 2300 and/or the system 500 or 1900. The communication module 2314 may communicate within the processor circuit 2300 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the leaflet gripping and slitting device) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

As will be readily appreciated by those having ordinary skill in the art after becoming familiar with the teachings herein, the leaflet gripping and slitting device advantageously permits a clinician (e.g., a heart surgeon) to grasp and immobilize a heart valve leaflet and then cut the leaflet with a repeatably straight incision that extends no farther than the tip of the gripper's jaws, and this presents minimal risk of damage to the heart wall or other adjacent tissues. Although intended for specific treatment of heart valve related diseases, the technology disclosed herein could be expanded to anywhere tissue needs to be remotely grasped and then cut, such as endoscopic, laparoscopic, intravascular, intraluminal, and/or robotic surgical procedures.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure.

Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A gripping and slitting device, comprising:
a catheter configured to be positioned within a body lumen of a patient;
a gripping assembly coupled to the catheter and comprising:
a first jaw;
a second jaw;
a hinge mechanism coupled to the first jaw and the second jaw and configured to rotate the first jaw relative to the second jaw between an open state and a closed state; and
a cutting assembly coupled to the gripping assembly and comprising:
at least one conductor; and
a cutting element configured to be energized by the at least one conductor.

2. The gripping and slitting device of claim 1,
further comprising a guide track disposed within at least one of the first jaw or the second jaw,
wherein the cutting assembly is configured to move along the guide track between a proximal portion and a distal portion of the gripping assembly.

3. The gripping and slitting device of claim 1,
wherein the first jaw is fixedly attached to the catheter, and
wherein the second jaw is rotatable relative to the first jaw via the hinge mechanism.

4. The gripping and slitting device of claim 1, wherein the cutting assembly is fixed to at least one of the first jaw or the second jaw.

5. The gripping and slitting device of claim 1, wherein the cutting element comprises a wire configured to be heated by electrical energy carried by the at least one conductor.

6. The gripping and slitting device of claim 5, wherein the wire comprises an exposed portion of the at least one conductor.

7. The gripping and slitting device of claim 1, further comprising a return electrode configured to be in contact with an exterior of the patient's body.

8. The gripping and slitting device of claim 1, wherein the cutting element comprises a plasma-generating electrode.

9. The gripping and slitting device of claim 1, wherein the hinge mechanism comprises a hinge pin and a pull wire.

10. The gripping and slitting device of claim 1,
wherein the body lumen comprises an aorta or a heart chamber of the patient,
wherein the gripping assembly is configured to grip a leaflet of a heart valve of the patient, and
wherein the cutting assembly is configured to slit at least a portion of the leaflet.

11. A system, comprising:
a power source;
an actuator;
a gripping and slitting device comprising:
a catheter configured to be advanced to a heart valve of a patient;
a gripping assembly coupled to the catheter and comprising:
a first jaw;
a second jaw;
a hinge mechanism coupled to the actuator, the first jaw, and the second jaw, wherein the hinge mechanism is configured to rotate the first jaw relative to the second jaw between an open state and a closed state, wherein the first jaw and the second jaw are configured to grip a leaflet of a heart valve in the closed state; and
a cutting assembly coupled to the gripping assembly and comprising:
at least one conductor coupled to the power source; and
a cutting element configured to slit the leaflet when energized by the at least one conductor.

12. The system of claim 11,
further comprising a guide track disposed within at least one of the first jaw or the second jaw,
wherein the cutting assembly is configured to move along the guide track between a proximal portion and a distal portion of the gripping assembly.

13. The system of claim 11,
wherein the first jaw is fixedly attached to the catheter, and
wherein the second jaw is rotatable relative to the first jaw via the hinge mechanism.

14. The system of claim 11, wherein the cutting assembly is fixed to at least one of the first jaw or the second jaw.

15. The system of claim 11, wherein the cutting element is a wire configured to be heated by electrical energy carried by the at least one conductor.

16. The system of claim 15, wherein the wire comprises an exposed portion of the at least one conductor.

17. The system of claim 11, further comprising a return electrode configured to be in contact with an exterior of the patient's body.

18. The system of claim 11, wherein the cutting element comprises a plasma-generating electrode.

19. The system of claim 11, wherein the hinge mechanism comprises a hinge pin and a pull wire.
